(19) United States
(12) Patent Application Publication
(10) Pub. No.: US 2016/0287637 A1
(43) Pub. Date: Oct. 6, 2016

(54) Synthetic composition and method for modulating emotion and mood disorders

(71) Applicant: GLYCOM A/S

(72) Inventors: Bruce McConnell, La Tour de Peilz (CH); Louise Kristine Vigsnæs, Kobenhavn NV (DK); Emma Salomonsson, Malmo (SE)

(21) Appl. No.: 15/183,456

(22) Filed: Jun. 15, 2016

Related U.S. Application Data
(63) Continuation-in-part of application No. 15/104,794, filed on Jun. 15, 2016.

(30) Foreign Application Priority Data
Dec. 8, 2014 (DK) PA 2014 70768

(51) Int. Cl. A61K 35/20

(52) U.S. Cl. CPC A61K 35/20

(57) ABSTRACT

The invention relates to a method for treating or preventing the development of emotion and/or mood disorders in a human individual.

### CROSS REFERENCE TO RELATED APPLICATIONS

This is a continuation-in-part of U.S. patent application Ser. No. 15/104,794, filed Jun. 15, 2016, which is a national stage filing in accordance with 35 U.S.C. §371 of PCT/DK2015/050385, filed Dec. 8, 2015, which claims the benefit of the priority of Denmark Patent Application No. PA 2014 70768, filed Dec. 8, 2014, the contents of each are incorporated herein by reference.

### FIELD OF THE INVENTION

This invention relates generally to compositions and methods for the treatment of emotion and mood disorders; for example, anxiety and depression. Compositions and methods of the inventions are particular useful for the treatment of depression in overweight, e.g. obese, individuals that have one or more metabolic risk factors.

### BACKGROUND TO THE INVENTION

Anxiety disorders are a group of mental disorders characterised by feelings of anxiety and fear (Diagnostic and Statistical Manual of Mental Disorders, American Psychiatric Association, 5th ed., 2013). Anxiety often occurs with other mental disorders, particularly mood or depressive disorders. Medication options are Benzodiazepines which are used for the short-term relief of severe anxiety, antidepressants, and Beta-blockers to reduce some of physical symptoms, such as rapid heartbeats and shaking.

Mood or depressive disorders are a group of disorders involving primary disturbances of mood. These include depression, major depressive disorder (MDD or clinical depression), dysthymia, and bipolar disorder. Patients suffering from depression exhibit feelings of sadness, low mood and an aversion to activity and this mood can affect a person's thoughts, behaviour, feelings and sense of well-being. A depressed person may feel sad, anxious, empty, hopeless, worried, helpless, worthless, guilty, irritable, hurt, or restless.

Major depressive disorder (MDD) is a disabling, severe mental disorder characterised by episodes of all-encompassing low mood, low self-esteem and loss of interest or pleasure in normally enjoyable activities. The illness tends to be chronic and repeated episodes are common. Other symptoms of MDD may include irritability or frustration, sleep disturbances, tiredness and lack of energy, changes in appetite, anxiety, agitation, restlessness, feelings of worthlessness or guilt, trouble thinking and concentrating, and unexplained physical problems, such as back pain or headaches. The disorder is a significant contributor to the global burden of disease and affects people in all communities across the world (Ferrari et al. PLoS Med. 10, e1001547 (2013)). MDD is a highly prevalent psychiatric disorder with twin studies revealing that up to 40% of MDD cases are genetically determined (Kendler et al. Psychol. Med. 36, 955 (2006)).

Although the exact causes of mood disorders are unknown, it is believed that a variety of factors may be involved, such as brain chemistry and physical brain differences, hormones, inherited traits and life events. This lack of knowledge of causation has made treatment difficult.

Many types of antidepressant medications are available to treat mood disorders that present with depression. Some available drugs include selective serotonin reuptake inhibitors (SSRIs), serotonin and norepinephrine reuptake inhibitors (SNRIs), norepinephrine and dopamine reuptake inhibitors (NDRIs), tricyclic antidepressants, monoamine oxidase inhibitors (MAOIs), and atypical antidepressants such as vortioxetine. However, despite the availability of numerous treatment options, individual response to antidepressant medication is suboptimal and variable. That is, not all individuals respond equally to a given antidepressant and some do not respond at all. As many as one half of patients do not receive adequate treatment and many respond partially or not at all to treatment. The presence of residual symptoms is also associated with a higher risk of recurrence, more chronic depressive episodes and a shorter duration between episodes. Guidelines for treatment recommend four possible strategies for managing non-response or partial response including: increasing the dose of the antidepressant drug; replacing the drug with a different antidepressant drug; augmenting the antidepressant therapy with a non-antidepressant agent; or combining the initial antidepressant with a second antidepressant.

Despite the lack of general efficacy, all of the available drugs have side effects with many having serious side effects. Therefore, safer, nutrition based option have also been investigated. One potential approach is the role of folate in central nervous system function because folate is a vitamin. Evidence suggests that folate can reduce depressive moods in certain patients; at least comparable to that of tricyclic antidepressants. Folate also appears to influence the rate of synthesis of tetrahydrobiopterin, a cofactor in the hydroxylation of phenylalanine and tryptophan, rate-limiting steps in the biosynthesis of dopamine, norepinephrine, and serotonin, neurotransmitters postulated to play a role in the pathogenesis of depression. In addition, methyltetrahydrofolate (MTHF) has been shown to bind to presynaptic glutamate receptors, where it may potentially modulate the release of other neurotransmitters, including the monoamines. However, folate appears to be best applicable to patients with certain genetic profiles (WO 2014/164882) and is probably not a solution for all patients.

Increasing evidence suggests that the intestinal microbiota also plays a key role in the generation of psychiatric disorders (Savignac et al. Neurogastroenterol. Motil. 26, 1615 (2014)). The intestinal microbiota consists of a vast bacterial community that resides primarily in the colon and lives in a symbiotic relationship with the host. The human gastrointestinal microbiota includes at least 1000 different species of bacteria, which collectively make up to 10¹⁴ bacterial cells, tenfold the number of human cells, and they encode 100-fold more unique genes than the human genome (Qin et al. Nature 464, 59 (2010)). A bidirectional neurohumoral communication system, known as the gut-brain axis, integrates the host gut and brain activities.

This has lead researchers to attempt using probiotics as a treatment option. Although data are limited, Lactobacillus and Bifidobacterium species have been shown to display potential therapeutic properties in psychiatric disorders (Dinan et al. Biol. Psychiatry 74, 720 (2013)). Also, early evidence indicates that some prebiotics may be another treatment option. The human milk oligosaccharides 3′-sialyllactose (3′-SL) and 6′-sialyllactose (6′-SL) support normal behavioural responses in mice during stressor exposure, potentially through effects on the gut microbiota-brain axis (Tarr et al. Brain Behay. Immun. 50, 166 (2015)).

Strongly overweight and obese individuals having one or more metabolic risk factors (the “unhealthy obese”) are at a higher risk of suffering from depression (Jokela et al. Mol. Psychiatry 19, 910 (2014)). These risk factors include dyslipidaemia, insulin resistance and/or impaired glucose control, hypertension, chronic inflammation, dysregulation of the hypothalamic-pituitary-adrenocortical axis, and excess visceral fat. At least some of these risk factors are related to intestinal microbiota.

Therefore, there remains a need for a generally safe and effective way for preventing or treating emotion and mood disorders, for example, anxiety and depression.

### SUMMARY OF THE INVENTION

In one aspect, this invention provides a synthetic composition for use in treating emotion and/or mood disorders, for example anxiety and/or depression, particularly in a patient having one or more of obesity coupled with a metabolic risk factor, stress, bacterial overgrowth, dysbiosis and an impaired mucosal barrier, characterised in that the composition contains an effective amount of one or more neutral human milk oligosaccharides (HMOs). The synthetic composition is preferably a nutritional composition.

In another aspect, this invention provides a method for treating an emotion and/or mood disorder patient, for example an anxiety and/or depression patient, particularly an individual having one or more of obesity coupled with a metabolic risk factor, stress, bacterial overgrowth, dysbiosis and an impaired mucosal barrier, the method comprising orally or enterally administering to the patient an effective amount of one or more neutral HMOs, preferably in the form of a synthetic composition. Preferably the abundance of bifidobacteria, more preferably a bifidobacterium of the B. adolescentis phylogenetic group, especially Bifidobacterium adolescentis and/or Bifidobacterium pseudocatenulatum, is increased in the colon of the patient with HMO therapy. Preferably one or more of bacterial overgrowth, dysbiosis, and/or impairment of mucosal barrier, is reduced in the patient. The method can be an adjunct treatment for a patient receiving other medication.

In a further aspect, this invention provides a method for maintaining an emotion and/or mood disorder patient in remission, for example an anxiety and/or depression patient, particularly a individual having one or more of obesity coupled with a metabolic risk factor, stress, bacterial overgrowth, dysbiosis, and/or an impaired mucosal barrier, the method comprising orally administering to the patient an effective amount of one or more neutral HMOs, preferably in the form of a synthetic composition.

The patient can be administered a higher amount, preferably 5 g to 10 g per day, of the one or more neutral HMOs for an initial treatment period, followed by a lower amount, preferably 1 g to 5 g per day, for a maintenance period. The initial treatment period can be 1 to 12 weeks. The maintenance period is at least 6 months.

In a further aspect, this invention provides one or more neutral HMOs, preferably in the form of a synthetic composition, for treating emotion and/or mood disorders, for example anxiety and/or depression, particularly in an individual having one or more of obesity coupled with a metabolic risk factor, stress, bacterial overgrowth, dysbiosis and an impaired mucosal barrier.

In a further aspect this invention provides one or more neutral HMOs, preferably in the form of a synthetic composition, for preventing development of emotion and/or mood disorders, for example anxiety and/or depression, particularly in an individual having one or more of obesity coupled with a metabolic risk factor, stress, bacterial overgrowth, dysbiosis and an impaired mucosal barrier.

Preferably the neutral HMO is selected from core HMOs and fucosyl HMOs, e.g. 2′-FL, 3-FL, DFL, LNT, LNnT, and LNFP-1. More preferably the HMO is a combination of one or more core HMOs and one or more fucosyl HMOs, for example 2′-FL and/or DFL and LNnT and/or LNT. The 2-'FL and/or DFL and LNnT and/or LNT may be present in a mass ratio of about 4:1 to 1:1; more preferably about 3:1 to 1:1.

### DETAILED DESCRIPTION OF THE INVENTION

In accordance with this invention, it has been surprisingly found that neutral human milk oligosaccharides (HMOs), advantageously 2′-FL, 3-FL, LNT, LNnT, LNT and/or DFL, are able to prevent development of and treat emotion and/or mood disorders, for example anxiety and depression, particularly in human individuals who are suffering from obesity coupled with a metabolic risk factor, stress, bacterial overgrowth, dysbiosis or an impaired mucosal barrier. It is believed that the HMOs can: (1) act as prebiotics to promote beneficial bacteria growth, especially bifidobacteria, and reduce bacterial overgrowth and dysbiosis; (2) act as decoys for pathogens by binding to them and thereby reduce/prevent binding of the pathogens to epithelial cells in the gastrointestinal tract; (3) act to reduce chronic mucosal inflammation; (4) reduce insulin resistance, and/or (5) repair damage to the mucosal barrier. By reducing chronic mucosal inflammation including reducing mast cell degranulation, and/or repairing damage to the mucosal barrier, the HMOs can also have beneficial effects on the enteric nervous systems of patients; potentially reducing anxiety and stress. Further, bifidobacteria, including Bifidobacterium adolescentis, are able to synthesis folate de novo, ensuring its constant bioavailability, and can secrete neuromodulators such as gamma-aminobutyric acid (GABA), a potent inhibitory neurotransmitter involved in reducing stress, anxiety and depression.

The intestinal bacteria may directly communicate with the central nervous system by way of the vagal sensory nerve fibres and the peripheral immune system. By altering the microbiota to increase bifidobacteria abundance, amongst other impacts, neutral HMOs may be capable of influencing neurotransmission in the paraventricular hypothalamus, the central nucleus of the amygdala, and the bed nucleus of the stria terminalis. All three of these regions are involved in the processing of emotions related to anxiety and mood.

### Terms and Definitions

The term “human individual” means a human subject of at least 3 years old. Interchangeably, the human individual of the invention is called “non-infant human” and “non-infant”. A human can be a child, a teenager, an adult or an elderly, preferably, the human is an individual of at least 3 years old that has an excess of body fat, more preferably, an individual whose excess body fat has accumulated to the extent that it may have a negative effect on health, i.e. an overweight or obese human individual. In some embodiments, the human individual is termed “patient” which means a human individual that has been diagnosed by a medical practitioner as having a disease.

The term “human milk oligosaccharide” or “HMO” preferably means a complex carbohydrate consisting of a small number, typically 3-10, of monosaccharide units attached to each other by an interglycosidic linkage that can be found in human breast milk and that can be in neutral or acidic form. More than about 200 different HMO structures are known to exist in human breast milk (Urashima et al.: Milk Oligosaccharides, Nova Biomedical Books, New York, 2011). Neutral HMOs are devoid of sialic acid, and can be core (non-fucosylated) and fucosylated oligosaccharides. Core HMOs consist of Glc, Gal and GIcNAc. Examples of core HMOs include lacto-N-tetraose (LNT), lacto-N-neotetraose (LNnT), lacto-N-neohexaose (LNnH) and lacto-N-hexaose (LNH). Fucosyl HMOs are fucosylated lactoses or fucosylated core HMOs such as 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose III (LNFP-III), fucosyl-para-lacto-N-neohexaose (F-pLNnH), lacto-N-difucohexaose I (LNDFH-I), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-lacto-N-hexaose III (FLNH-III) and fucosyl-para-lacto-N-neohexaose (F-pLNnH).

The HMOs can be isolated or enriched by well-known processes from milk(s) secreted by mammals including, but not limited to human, bovine, ovine, porcine, or caprine species. The HMOs can also be produced by well-known processes using microbial fermentation, enzymatic processes, chemical synthesis, or combinations of these technologies. As examples, using chemistry LNnT can be made as described in WO 2011/100980 and WO 2013/044928, LNT can be synthesized as described in WO 2012/155916 and WO 2013/044928, a mixture of LNT and LNnT can be made as described in WO 2013/091660, 2′-FL can be made as described in WO 2010/115934 and WO 2010/115935, 3-FL can be made as described in WO 2013/139344, and mixtures of human milk oligosaccharides can be made as described in WO 2012/113405. As examples of enzymatic production, fucosylated oligosaccharides can be made as described in WO 2012/127410, and advantageously diversified blends of human milk oligosaccharides can be made as described in WO 2012/156897 and WO 2012/156898. With regard to biotechnological methods, WO 01/04341 and WO 2007/101862 describe how to make core human milk oligosaccharides optionally substituted by fucose using genetically modified E. coli. If it is desired to additionally include acidic HMO's, these can be obtained as described in WO 2012/113404, WO 2012/007588, WO 01/04341 and WO 2007/101862.

The terms “microbiota”, “microflora” and “microbiome” preferably mean a community of living microorganisms that typically inhabits a bodily organ or part. The most dominant members of the gastrointestinal microbiota include microorganisms of the phyla of Firmicutes, Bacteroidetes, Actinobacteria, Proteobacteria, Synergistetes, Verrucomicrobia, Fusobacteria, and Euryarchaeota. At genus level the dominant microorganisms are Bacteroides, Faecalibacterium, Bifidobacterium, Roseburia, Alistipes, Collinsella, Blautia, Coprococcus, Ruminococcus, Eubacterium and Dorea; and at species level common species are Bacteroides uniformis, Alistipes putredinis, Parabacteroides merdae, Ruminococcus bromii, Dorea longicatena, Bacteroides caccae, Bacteroides thetaiotaomicron, Eubacterium hallii, Ruminococcus torques, Faecalibacterium prausnitzii, Ruminococcus lactaris, Collinsella aerofaciens, Dorea formicigenerans, Bacteroides vulgatus and Roseburia intestinalis. In some instances, the gastrointestinal microbiota includes the mucosa-associated microbiota, which is located in or attached to the mucus layer covering the epithelium of the gastrointestinal tract, and luminal-associated microbiota, which is found in the lumen of the gastrointestinal tract.

The term “bifidobacterium of the B. adolescentis phylogenetic group” means a bacterium selected from a group consisting of Bifidobacterium adolescentis, Bifidobacterium angulatum, Bifidobacterium catenulatum, Bifidobacterium pseudocatenulatum, Bifidobacterium kashiwanohense, Bifidobacterium dentum and Bifidobacterium stercoris (Duranti et al. Appl. Environ. Microbiol. 79, 336 (2013), Bottacini et al. Microbial Cell Fact. 13:S4 (2014)).

The term “metabolic risk factor” means a factor in an obese human which increases the risk of the patient developing metabolic co-morbidities; for example, type 2 diabetes and cardiovascular disease. Examples of metabolic risk factors include dyslipidaemia, insulin resistance and/or impaired glucose control, hypertension, chronic inflammation, dysregulation of the hypothalamic-pituitary-adrenocortical axis, and excess visceral fat.

The term “emotion or mood disorder” relates to a category of illnesses that describe a serious change in mood. Illness under mood disorders include: major depressive disorder (also known as depression), bipolar disorder (mania—euphoric, hyperactive, over inflated ego, unrealistic optimism), persistent depressive disorder (long lasting low grade depression), cyclothymia (a mild form of bipolar disorder), anxiety (a feeling of worry, nervousness, or unease about something with an uncertain outcome)and SAD (seasonal affective disorder).

The term “obese human individual” means that a human individual that has a body mass index (BMI), a measurement obtained by dividing the individual's weight by the square of the individual's height, over 30 kg/m², with the range 25-30 kg/m² defined as overweight. The term “strong overweight” relates to BMI of 28 to 30 kg/m².

The term “bacterial overgrowth” means bacterial overgrowth syndrome (BOS) (interchangeably termed small intestinal bacterial overgrowth (SIBO) or small bowel bacterial overgrowth syndrome (SBBOS)) relates to clinical manifestations that occur when the normally low number of bacteria that inhabit the stomach, duodenum, jejunum, and proximal ileum significantly increases or becomes overtaken by other pathogens Bacterial overgrowth made be determined by a number of techniques, with the gold standard being an aspirate from the jejunum that grows in excess of 10⁵ bacteria per milliliter. Patients with bacterial overgrowth typically develop symptoms including nausea, bloating, vomiting, diarrhea, malnutrition, weight loss and malabsorption. The term “dysbiosis” (also interchangeably called dysbacteriosis) is a term for a microbial imbalance on or inside the body. In the present context dysbiosis is a condition in the digestive tract that can be associated with an illnesses, such as irritable bowel syndrome, inflammatory bowel disease, chronic fatigue syndrome, obesity, cancer, bacterial vaginosis, and colitis.

The term “oral administration” preferably means any conventional form for the oral delivery of a composition to a patient that causes the deposition of the composition in the gastrointestinal tract (including the stomach) of the patient. Accordingly, oral administration includes swallowing of composition by the patient, enteral feeding through a naso-gastric tube, and the like.

The term “effective amount” preferably means an amount of a composition that provides a neutral human milk oligosaccharide in a sufficient amount to render a desired treatment or prevention outcome in a patient. An effective amount can be administered in one or more doses to the patient to achieve the desired treatment or prevention outcome.

The term “preventing (development) of emotion or mood disorder” in the present context means eliminating or minimising a chance of development of an emotion or mood disorder. Both primary and secondary prevention are thus contemplated. The primary prevention means preventing a an emotion or mood disorder before it occurs, and the secondary prevention means preventing additional attacks of an emotion or mood disorder after the first attack has occurred.

The term “synthetic composition” means a composition which is artificially prepared and preferably means a composition containing at least one compound that is produced ex vivo chemically and/or biologically, e.g. by means of chemical reaction, enzymatic reaction or recombinantly. In some embodiments a synthetic composition of the invention may be, but preferably is not, identical with a naturally occurring composition. The synthetic composition of the invention typically comprises one or more compounds, advantageously HMOs, that are capable of preferentially increasing the abundance of bifidobacteria, in particular Bifidobacterium of the following species: Bifidobacterium longum, Bifidobacterium bifidum, and/or members of the phylogenetic Bifidobacterium adolescentis group. In some embodiments, the synthetic composition may comprise one or more compounds or components other than HMOs that may have an effect on bifidobacteria of a human subject microbiota in vivo, e.g. non-digestible oligosaccharides or prebiotics. Also in some embodiments, the synthetic compositions may comprise one or more nutritionally or pharmaceutically active components which do not affect adversely the efficacy of the above mentioned compounds. Some non-limiting embodiments of a synthetic composition of the invention are also described below.

The synthetic composition comprising one or more neutral human milk oligosaccharides can take any suitable form. For example, the composition can be in the form of a nutritional composition which contains other macronutrients such as proteins, lipids or other carbohydrates. The synthetic composition can also be an incomplete nutritional composition in unit dosage form or pharmaceutical composition. In one embodiment, the synthetic compositions contain one or more core HMOs and one or more fucosyl HMOs. In a preferred embodiment, the synthetic composition contains 2′-FL and/or DFL and LNnT and/or LNT.

### Nutritional Compositions

A nutritional composition can contain sources of protein, lipids and/or digestible carbohydrates and can be in powdered or liquid forms. The composition can be designed to be the sole source of nutrition or a nutritional supplement. For emotion or mood disorder patients, a nutritional supplement is preferred; especially a supplement which can form a meal or snack replacement. Preferably the nutritional composition is lactose-reduced or, better yet, lactose-free.

Suitable protein sources include milk proteins, soy protein, rice protein, pea protein and oat protein, or mixtures thereof. Milk proteins can be in the form of milk protein concentrates, whey protein or casein, or mixtures of both. Soy, rice, pea and oat protein can be in the form or protein isolated. The protein can be whole protein or hydrolysed protein, either partially hydrolysed or extensively hydrolysed. The protein can provide about 5% to about 50%, preferably about 10% to 30%, of the energy of the nutritional composition. The protein source preferably is not a source of carbohydrates such as lactose. Therefore, if a milk protein is used as the protein source, the milk protein is preferably lactose-reduced or lactose-free.

Suitable digestible carbohydrates include maltodextrin, hydrolysed or modified starch or corn starch, glucose polymers, corn syrup, corn syrup solids, tapioca, sucrose, and glucose, or mixtures thereof. Generally digestible carbohydrates provide about 35% to about 75%, preferably about 45% to 70%, of the energy of the nutritional composition. Preferably the digestible carbohydrate is free from lactose.

Suitable lipids include rapeseed oil, sunflower seed oil, palm oil, soy oil, milk fat, corn oil and soy lecithin. Long-chain poly unsaturated fatty acids (LC-PUFA), especially omega-3 fatty acids such as docosahexaenoic acid (DHA), can be included in the lipid source because they have anti-inflammatory properties. Suitable sources of LC-PUFA are plant oils, marine plankton oils, fungal oils, and fish oils. The lipid source can also include medium chain triglycerides (MCT). Fractionated coconut oils are a suitable source of medium chain triglycerides. The lipid source preferably provides about 5% to about 25% of the energy of the nutritional composition; for example, about 10% to 20 %.

The nutritional composition preferably also includes vitamins and minerals. If the nutritional composition is intended to be a sole source of nutrition, it preferably includes a complete vitamin and mineral profile. Examples of vitamins include Vitamins A, B-complex (such as B1, B2, B6 and B12), C, D, E and K, niacin and acid vitamins such as pantothenic acid and folic acid and biotin. Examples of minerals include calcium, iron, zinc, magnesium, iodine, copper, phosphorus, manganese, potassium, chromium, molybdenum, selenium, nickel, tin, silicon, vanadium and boron.

The nutritional composition can also include a carotenoid such as lutein, lycopene, zeaxanthin, and beta-carotene. The total amount of carotenoid included can vary from about 0.001 μg/ml to about 10 μg/ml. Lutein can be included in an amount of from about 0.001 μg/ml to about 10 μg/ml, preferably from about 0.044 μg/ml to about 5 μg/ml of lutein. Lycopene can be included in an amount from about 0.001 μg/ml to about 10 μg/ml, preferably about 0.0185 μg/ml to about 5 μg/ml of lycopene. Beta-carotene can comprise from about 0.001 μg/ml to about 10 μg/ml, for example about 0.034 μg/ml to about 5 μg/ml of beta-carotene.

The nutritional composition can also contain various other conventional ingredients such as preservatives, emulsifying agents, thickening agents, buffers, fibres and probiotics, especially probiotics which can help to reduce symptoms in patients (e.g. Lactobacillus casei strain Shirota, B. infantis 35624, B. animalis subsp. lactis BB-12, B. lactis Bi-07, L. rhamnosus GG, L. rhamnosus Lc705, L. plantarum DSM 9843, L. plantarum CECT7484, L. plantarum CECT7485, L. acidophilus NCFM, L. fermentum CECT5716, B. breve Bb99, Propionibacterium freundenreichii ssp. Shermanii JS, P. acidilactici CECET7483, Streptococcus faecium), antioxidant/anti-inflammatory compounds including tocopherols, caroteinoids, ascorbate/vitamin C, ascorbyl palmitate, polyphenols, glutathione, and superoxide dismutase (melon), other bioactive factors (e.g. growth hormones, cytokines, TFG-(β), colorants, flavours, and stabilisers, lubricants, and so forth.

The nutritional composition can be in the form of a soluble powder, a liquid concentrate, or a ready-to-use formulation. Various flavours, fibres and other additives can also be present.

The nutritional compositions can be prepared by any commonly used manufacturing techniques for preparing nutritional compositions in solid or liquid form. For example, the composition can be prepared from various feed solutions. A protein-in-fat feed solution can be prepared by heating and mixing the lipid source and then adding an emulsifier (e.g., lecithin), fat soluble vitamins, and at least a portion of the protein source while heating and stirring. A carbohydrate feed solution is also prepared by adding minerals, trace and ultra trace minerals, thickening or suspending agents to water while heating and stirring. The resulting solution is held for 10 minutes with continued heat and agitation before adding carbohydrates (e.g., the HMOs and digestible carbohydrate sources). The resulting feed solutions are then blended together while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavours are added, and water is added to achieve the desired total solid level.

For a liquid product, the resulting solution can then be aseptically packaged to form an aseptically packaged nutritional composition. In this form, the nutritional composition can be in ready-to-feed or concentrated liquid form. Alternatively, the composition can be spray dried and processed and packaged as a reconstitutable powder.

When the nutritional product is a ready-to-feed nutritional liquid, the total concentration of HMOs in the liquid, by weight of the liquid, is from about 0.002%to about 3.0%, including from about 0.005% to about 2%, including from about 0.05% to about 1.0%. When the nutritional product is a concentrated nutritional liquid, the total concentration of HMOs in the liquid, by weight of the liquid, is from about 0.004% to about 6.0%, including from about 0.01% to about 4.0%, including from about 0.1% to about 2.0%.

According to the invention, any synthetic composition described herein comprises an effective amout of one or more HMOs, e.g. 2′-FL, LNnT, etc., or an effective amount of a mixture of two or more HMOs, e.g. 2′-FL and LNnT, etc. In one embodiment, the synthetic composition comprises a mixture of 2′-FL and LNnT, preferably, the mass ration between 2′-FL and LNnT (i.e. 2′-FL:LNnT) in the mixture is in the range from 4:1 to 1:1.

### Unit Dosage Forms

The synthetic composition of this invention can also be in a unit dosage form such as a capsule, tablet or sachet. For example, the composition can be formulated into single serve sachets containing the HMO's; especially if higher doses are to be administered (more than 3 g). Alternative the composition can be in a tablet form comprising the human milk oligosaccharides, and one or more additional components to aid formulation and administration, such as diluents, excipients, antioxidants, lubricants, colorants, binders, disintegrants, and the like.

Suitable diluents, excipients, lubricants, colorants, binders, and disintegrants include polyethylene, polyvinyl chloride, ethyl cellulose, acrylate polymers and their copolymers, hydroxyethyl-cellulose, hydroxypropylmethyl-cellulose (HPMC), sodium carboxymethylcellulose, polyhydroxyethyl methacrylate (PHEMA), polyvinyl alcohol (PVA), polyvinyl pyrrolidone (PVP), polyethylene oxide (PEO), or polyacrylamide (PA), carrageenan, sodium alginate, polycarbophil, polyacrylic acid, tragacanth, methyl cellulose, pectin, natural gums, xanthan gum, guar gum, karaya gum, hypromellose, magnesium stearate, microcrystalline cellulose, and colloidal silicon dioxide. Suitable antioxidants are vitamin A, carotenoids, vitamin C, vitamin E, selenium, flavonoids, polyphenols, lycopene, lutein, lignan, coenzyme Q10 (“CoQlO”) and glutathione.

The unit dosage forms, especially those in sachet form, can also include various nutrients including macronutrients.

### Administration Dosing

For improving emotion and mood disorders in a human individual, in particular in a patient, especially those suffering from obesity coupled with a metabolic risk factor, stress, bacterial overgrowth, dysbiosis and/or an impaired mucosal barrier, the amount of neutral HMO(s) required to be administered to the patient will vary depending upon factors such as the risk and severity of the disease, the age of the patient, the form of the composition, and other medications being administered to the patient. However, the required amount can be readily determined by a medical practitioner and would generally be in the range of about 20 mg to about 30 g per day, preferably about 50 mg to about 20 g per day, or from about 100 mg to about 15 g per day, in certain embodiments from about 500 mg to about 10 g per day, preferably from about 1 g to about 7.5 g per day. During an initial treatment phase, the dosing can be higher; for example, 100 mg to 30 g per day, preferably 500 mg to 15 g per day, more preferably 1 g to 10 g per day, in certain embodiments 2.5 g to 7.5 g per day. During a secondary prevention phase, the dosing can be reduced; for example, to 20 mg to 20 g per day, preferably 100 mg to 10 g per day, more preferably 500 g to 7.5 g per day, in certain embodiments 750 mg to 5 g per day.

### Embodiments of the invention

Different embodiments of the invention include methods of treatment and methods of prevention of an emotion or mood disorder. In particular, in one embodiment the invention relates to a method for treating emotion and/or mood disorders in a patient, particularly in a patient having one or more of obesity coupled with a metabolic risk factor, stress, bacterial overgrowth, dysbiosis and an impaired mucosal barrier. In another embodiment, the invention relates to preventing development of emotion and/or mood disorders in a human individual, in particular in a patient having one or more of obesity coupled with a metabolic risk factor, stress, bacterial overgrowth, dysbiosis and an impaired mucosal barrier, the method comprising orally administering to the patient an effective amount of one or more neutral HMOs, preferably in the form of a synthetic composition. Preferably, the amount of the one or more HMO is effective to increase the abundance of bifidobacteria in the gastro-intestinal tract of the treated individual, preferably a bifidobacterium of the B. adolescentis phylogenetic group, especially Bifidobacterium adolescentis and/or Bifidobacterium pseudocatenulatum. The individual is preferably an obese human individual who is suffering from obesity coupled with a metabolic risk factor, stress, bacterial overgrowth, dysbiosis or an impaired mucosal barrier.

Preferably, methods of the invention relate to treating or preventing symptoms of anxiety and/or depression. According to the invention, the later symptoms are improved in patients treated with compositions disclosed herein. Preferably, a patient is administered an amount of one or more neutral HMO in the range from about 5 g to about 10 g per day as an initial treatment period, followed by a lower amount, preferably 1 g to 5 g per day, for a maintenance period.

Another embodiment of the invention relates to one or more neutral HMOs for treating
emotion and/or mood disorders, or
preventing development of emotion and/or mood disorders

in a patient, particularly in a patient having one or more of obesity coupled with a metabolic risk factor, stress, bacterial overgrowth, dysbiosis and an impaired mucosal barrier.

The neutral HMO in any of the above embodiments may be a single neutral HMO or a mixture of any neutral HMOs suitable for the purpose of the invention. Preferably, the invention relates to a mixture of neutral HMOs. More preferably, the mixture comprises at least a first HMO and at least a second HMO, wherein the first HMO is a fucosylated neutral HMO and the second HMO is a core HMO. Particularly, the mixture of HMOs may contain a fucosylated HMO selected from the list consisting of 2′-FL, 3-FL, DFL, LNFP-I, LNFP-II, LNFP-III, LNFP-V, LNDFH-I, LNDFH-II, LNDFH-III, FLNH-I, FLNH-II, FLNnH, FpLNH-I and F-pLNnH II, and a core HMO selected from the list consisting of LNT, LNnT, LNH, LNnH, pLNH and pLNnH. Preferably, the mixture of neutral HMOs contains a fucosylated HMO selected from the list consisting of 2′-FL, 3-FL and DFL, and a core HMO selected from the list consisting of LNT and LNnT; advantageously the mixture comprises 2′-FL and LNnT and/or LNT. In some embodiments, the mixture of HMOs essentially consists of two neutral HMOs, e.g. a fucosylated HMO selected from the list consisting of 2′-FL, 3-FL, DFL, LNFP-I, LNFP-II, LNFP-III, LNFP-V, LNDFH-I, LNDFH-II, LNDFH-III, FLNH-I, FLNH-II, FLNnH, FpLNH-I and F-pLNnH II, and a core HMO selected from the list consisting of LNT, LNnT, LNH, LNnH, pLNH and pLNnH. Preferably, the mixture essentially consists of a fucosylated HMO selected from the list consisting of 2′-FL, 3-FL and DFL, and a core HMO selected from the list consisting of LNT and LNnT; in one preferred embodiment the mixture essentially consists of 2′-FL and LNnT, in another preferred embodiment the mixture essentially consists of 2′-FL and LNT.

### EXAMPLES

Examples are now described to further illustrate the invention:

### Example 1

### Human Trial

A total of 40 male and female patients are recruited to participate in the study. The patients are screened from a pool of diagnosed patients with depression and anxiety. After a screening visit and run-in period of 1-2 weeks, the patients are selected. The patients are randomized into two groups, each of 20 patients, with one group consuming the treatment product and one group the placebo product for 8 weeks. The treatment product contains 5 grams of a combination of 2′-FL and LNnT while the control product contains 5 grams glucose. Both products are in powder form in a unit dosage container.

The patients are eligible to participate if they are aged 18-65, meet the formal diagnostic criteria for depression and anxiety, and suitable to complete a two month trial. All recruited patients are able and willing to understand and comply with the study procedures. Patients are excluded if: they have participated in a clinical study one month prior to screening visit; they have abnormal results in the screening tests which are clinically relevant for study participation; they are suffering for a severe disease such as malignancy, diabetes, severe coronary disease, kidney disease, neurological disease, or severe psychiatric disease or any condition which can confound the results of the study; used highly dosed probiotic supplements (yoghurt allowed) for 3 months prior to the study; consumed antibiotic drugs 3 months prior to the study; consumed on a regular basis any medication that might interfere with symptom evaluation 2 weeks prior to the study; and pregnant or lactating.

At the initial visit (screening), each patient is given both written and oral information about the study and the patient is asked to sign an informed consent form. Patients are evaluated by a full review of clinical history. A blood sample for eligibility analysis is collected. A talk through of the electronic questionnaires (GSRS, QoL, BDI, BAI and BSFS) is performed to familiarise the patients with the electronic system, and equipment for faecal sampling is distributed to each patient. Patients are instructed to keep their samples in the freezer until the next visit.

At the second visit (beginning of intervention), eligibility criteria are checked and eligible subjects are randomised to the two arms in the trial. Symptoms (as measured by GSRS, QoL, BDI, BAI and BSFS scales) are assessed. Trial supplementation is distributed along with instructions on use of an electronic compliance diary. The faecal samples are collected and equipment for collecting new samples are distributed. Patients are reminded not to change their usual diet during the study.

Blood samples are collected for biomarker studies and biobanking. The serum from the blood samples is transferred to cryotubes and stored at −80° C. The following biomarkers are measured TNF-α, IL-1β, IL-8, IL-6, IL-12, IL-10, MIP-1β, hs-CRP, lipopolysaccharide binding protein, tryptase, antiflagellin, zonulin, histamine, prostaglandin 2, and cortisol. The faecal samples are stored at −80° C. until analysis. Microbiological analysis is performed on the faecal samples using the 16 S rRNA gene sequence.

The study runs for 8 weeks with the patients consuming either a placebo or a treatment product daily. Patients are instructed to consume the products in the morning with breakfast. Compliance is monitored through the interactive internet enabled system. The patients also use the system to record:
Bristol Stool Form Scale (BSFS) information,
Gastrointestinal Symptom Rating Scale (GSRS) information. This questionnaire includes 15 items covering five dimensions (abdominal pain, indigestion, reflux, diarrhoea, constipation) and uses a seven-graded Likert scale,
Quality of life (QoL) information,
Beck Depression Inventory (BDI) and the Beck Anxiety Inventory (BAI) information.

4 weeks after commencement, there is an intermediate check. A physical examination is done and symptoms (as measured by GSRS, BSFS, QoL, BDI and BAI scales etc.) are reassessed. Faecal samples and blood samples are collected and analysed as before, and equipment for collection of new faecal samples are distributed.

At the end of the intervention (8 weeks), each patient has a visit with the medical team. A physical examination is done and symptoms (as measured by GSRS, BSFS, QoL, BDI and BAI scales etc.) are reassessed. Trial supplementation products are collected to check compliance.

Faecal samples and blood samples are collected and analysed as before.

Any patients who indicate any adverse events during the study are invited for a final visit to asked about any adverse events. This visit may be completed via telephone.

The treatment patients report a reduction in anxiety, a reduction in depression and an improvement in stress. The blood biomarker analysis indicates that the treatment patients have reduced levels of inflammatory markers, reduced gut permeability indicating an improved mucosal barrier, and reduced evidence of mast cell degranulation. The faecal analysis indicates that the treatment patients have reduced levels of bacterial overgrowth/dysbiosis and a higher level of bifidobacteria, especially bifidobacterium of the B. adolescentis phylogenetic group, especially Bifidobacterium adolescentis and/or Bifidobacterium pseudocatenulatum.

### Example 2

### Nutritional Composition

A ready to feed nutritional composition is prepared from water, maltodextrin, corn syrup, sugar, milk protein concentrate, vegetable oil (canola, high oleic sunflower and corn), soy protein isolate, acacia gum, flavours, HMOs, potassium citrate, magnesium phosphate, cellulose gel and gum, calcium carbonate, sodium ascorbate, soy lecithin, choline bitartrate, calcium phosphate, alpha-tocopheryl acetate, ascorbic acid, carrageenan gum, ferric pyrophosphate, flavours, sweeteners (Stevia), vitamin A palmitate, niacinamide, vitamin D3, calcium pantothenate, manganese sulphate, copper sulphate, pyridoxine hydrochloride, thiamine hydrochloride, beta carotene, riboflavin, chromium chloride, folic acid, biotin, potassium iodide, phytonadione, sodium selenite, sodium molybdate, vitamin B12.

The composition provides a nutritional supplement which is a good source of protein, low in fat, vitamins, minerals and antioxidants. Further, the composition contains HMOs which are able to promote the growth of beneficial intestinal bacteria, modulate chronic inflammation, improve mucosal barrier integrity and reduce anxiety and depression.

### Example 3

### Capsule Composition

A capsule is prepared by filling about 1 g of neutral HMO into a 000 gelatine capsule using a filing machine. The capsules are then closed. The neutral HMO are in free flowing, powder form.

1. A method for
treating emotion and/or mood disorders, or
preventing development of emotion and/or mood disorders
in a human individual, the method comprising orally administering to the human an effective amount of one or more neutral HMOs.

2. The method according to claim 1, wherein the human is an obese patient having one or more of obesity coupled with a metabolic risk factor, stress, bacterial overgrowth, dysbiosis and an impaired mucosal barrier.

3. The method according to claim 1, wherein the emotion and/or mood disorder is anxiety and/or depression.

4. The method according to claim 3, wherein symptoms of anxiety and/or depression are improved.

5. The method according to claim 1, wherein the more neutral HMOs comprises a core HMO and a fucosylated HMO.

6. The method according to claim 5, wherein the core HMO is selected from LNT and LNnT, and the fucosylated HMO is selected from 2′-FL, 3-FL and DFL.

7. The method according to claim 6, wherein the HMOs comprise any of the combinations of 2′-FL and LNnT, 2′-FL and LNT, or 2′-FL, LNT and LNnT.

8. The method according to claim 6, wherein the HMOs consist essentially of any combinations of 2′-FL and LNnT, or 2′-FL and LNT, or 2′-FL, LNT and LNnT.

9. The method according to claim 7 or 8, wherein, in a 2′-FL and LNnT combination, the 2′-FL and LNnT are present in a mass ratio of 4:1 to 1:1.

10. The method according to claim 1, wherein the human is administered an amount of 5 g to 10 g of the one or more neutral HMOs per day for an initial treatment period, followed by an amount of 1 g to 5 g of said HMOs per day for a maintenance period.

11. The method according to claim 1, wherein the one or more neutral HMOs are formulated in a synthetic composition.

12. The method according to claim 11, wherein the synthetic composition is a unit dosage form.

13. The method according to claim 1, wherein abundance of bifidobacteria is increased in the human.

14. The method according to claim 13, wherein the bifidobacterium is a bifidobacterium of the phylogenetic Bifidobacterium adolescentis phylogenetic group.

15. The method according to claim 14, wherein the bifidobacterium of the phylogenetic Bifidobacterium adolescentis phylogenetic group is Bifidobacterium adolescentis and/or Bifidobacterium pseudocatenulatum.
